(19) United States
(12) Patent Application Publication
(10) Pub. No.: US 2026/0287576 A1
(43) Pub. Date: Sep. 24, 2026

(54) POTENCY ASSAY MATRIX FOR NEURONAL CELLS

(71) Applicant: Aspen Neuroscience, Inc., San Diego, CA (US)

(72) Inventors: Daniel Fremgen, San Diego, CA (US); Candace Lynch, San Diego, CA (US)

(21) Appl. No.: 19/206,993

(22) Filed: May 13, 2025

Related U.S. Application Data
(60) Provisional application No. 63/646,994, filed on May 13, 2024.

(51) Int. Cl. G01N 33/50; A61K 35/30; C12N 5/0797; C12Q 1/527; G01N 30/02; G01N 30/72; G01N 30/88; G01N 33/94

(52) U.S. Cl. CPC G01N 33/5058; A61K 35/30; C12N 5/0623; C12Q 1/527; G01N 30/7233; G01N 33/5023; G01N 33/5038; G01N 33/9413; C12N 2506/45; G01N 2030/027; G01N 2030/8813; G01N 2333/988; G01N 2500/10; G01N 2800/245

(57) ABSTRACT

The invention provides, in some embodiments, a potency assay matrix for assessing potency of dopaminergic neuronal progenitor cells (DANPCs) that are intended for use in treating a neurodegenerative disease such as Parkinson's Disease. The potency assay matrix can include a bioassay for L-DOPA decarboxylase (DDC) activity. In addition to the DDC activity assay matrix, the potency assay matrix may also include one or more additional mechanism of action-based assays such as bioinformatics-based assays for determining whether the DANPCs are dopaminergic neuronal progenitor cells, for determining whether the DANPCs are likely to engraft when implanted into a subject brain, and for determining whether neuronal cells that are derived from the DANPCs are likely to produce dopamine after implantation.

### CROSS-REFERENCE TO RELATED APPLICATIONS

This application claims priority to U.S. Provisional Application No. 63/646,994, filed May 13, 2024, which application is incorporated herein by reference for all purposes.

### BACKGROUND OF THE INVENTION

### Field

The present disclosure relates to methods for assessing characteristics, such as potency and function, of an in vitro population of neuronal cells, such as neuronal progenitor cells, that are to be used for implantation into a subject in need of treatment for a neurodegenerative disease such as Parkinson's Disease.

### Background

Parkinson's Disease leads to debilitating motor complications and currently no restorative treatments are available. Parkinson's disease trails only Alzheimer's disease as the most common neurodegenerative disorder, affecting about 0.3% of the general population and 1-2% of the population over age 65. The prevalence of PD is expected to double or triple as the developed world population ages. Cha et al. (2023) J. Mov. Disord. 16:22-41; Rong et al. (2021) Neurology 97: e1986-e1993; Dorsey and Bloem (2018) JAMA Neurol. 75:9-10; de Lau and Breteler (2006) Lancet Neurol. 5:525-535).

Dopamine deficiency resulting from progressive loss of dopaminergic neurons in the substantia nigra is a common characteristic of PD. By the time of diagnosis, patients have already experienced significant nigrostriatal degeneration. Currently available treatments, such as dopamine replacement therapy (e.g., with L-dopa or dopamine agonists) benefit some patients, but have a limited therapeutic window due to side effects and decreasing efficacy. Cha et al., Weiss et al. (1971) Lancet. 1:1016-1017; Kang and Fahn (1988) Ration. Drug Ther. 22:1-7.

Cell replacement therapies that aim to restore lost dopamine producing neurons are currently in development. One such approach is to use a differentiation to guide the differentiation of pluripotent stem cells towards a dopaminergic neuronal cell fate (see, e.g., Telias (2023) Neural Regen. Res. 18:1273-1274 for a review of various protocols). These protocols often involve the sequential application of growth factors and small molecules that mimic the developmental cues encountered by dopaminergic neurons during embryogenesis. For example, inhibition of the TGFβ signaling pathway and activation of the sonic hedgehog (SHH) pathway have been shown to promote dopaminergic differentiation. See, e.g., Chambers et al. (2009) Nature Biotechnol. 27:275-280; US Patent Publication 2003/0059010. Signaling pathways such as SHH, fibroblast growth factor (FGF), Wnt, and Notch play critical roles in dopaminergic specification and maturation. The sequential activation and inhibition of these pathways are often integrated into differentiation protocols to mimic the spatiotemporal signaling dynamics observed during embryonic development. Transcription factors such as LMX1A, FOXA2 and NURR1, which are key regulators of dopaminergic neuron development have also been used to differentiate stem cells into dopaminergic neuronal cells (Kriks et al (2011) Nature 480:547-551).

While many differentiation protocols can generate cells expressing dopaminergic markers, using such cells for implantation into a subject remains a challenge. As an example, preparing the neuronal progenitor cells is often a lengthy process that requires specialized equipment and stringent control of conditions to avoid, for example, contamination of the cells. Such equipment and controls are often not present in proximity to the clinical site, however, so it is desirable to prepare the cells at a central facility and preserve them for storage, and eventual transport to the clinical site and subsequent implantation. Cryopreservation is one such method by which neuronal progenitor cells can be prepared at a central facility, preserved, transported to the treatment clinic, and used to treat a subject.

Improved methods are needed, however, for determining whether such cells are potent, and maintain potency after cryopreservation and subsequent thawing. In particular, assays are needed for assessing the potency and health of each batch of neuronal progenitor cells after manufacture and/or cryopreservation to ensure that each batch will produce the expected response. Often, such assays must meet strict governmental regulatory requirements for release of a pharmaceutical composition that are set by agencies such as the U.S. Food and Drug Administration (the FDA), the European Medicines Agency (the EMA), and the Japanese Pharmaceuticals and Medical Devices Agency (PMDA). The FDA, for example, requires that cell therapy products have a potency test for licensure, and states that potency tests should be based on the product's mechanism of action (MOA) (Draft Guidance for Industry-Potency Assurance for Cellular and Gene Therapy Products, US Food and Drug Administration (December 2023). https://www.fda.gov/regulatory-information/search-fda-guidance-documents/potency-assurance-cellular-and-gene-therapy-products). For cell therapies that have a complex spectrum of mechanisms, it is desirable to have a potency assay matrix that can quantitatively and accurately characterize the various important functions of the cells after they have matured. The present invention fulfils these and other needs.

### SUMMARY OF THE INVENTION

In some embodiments, the invention provides methods of assessing potency of a composition comprising a population of neuronal progenitor cells intended for implantation into a subject in need of treatment for a neurodegenerative disease. These methods involve assessing potency of the population of neuronal progenitor cells by determining an L-dihydroxyphenylalanine (DOPA) decarboxylase (DDC) activity level produced by the neuronal progenitor cells, wherein a DDC activity level that is elevated relative to that produced by a population of reference cells is indicative of sufficient potency for implantation.

In some embodiments, the methods further involve releasing the population of neuronal progenitor cells for use in treating the subject if the potency of the population of neuronal progenitor cells meets a regulatory potency requirement for release. Such regulatory potency requirements include those set by the US Food and Drug Administration or the European Medicines Agency, and other state, national, and international regulatory agencies.

In some embodiments, the methods involve determining a DDC activity level in medium after culturing a population of neuronal progenitor cells. In other embodiments, the methods involve determining the activity level of DDC in a cell lysate obtained from the population of neuronal progenitor cells. In some embodiments, the DDC activity level is determined in the medium after culturing the population of neuronal progenitor cells.

In some embodiments, the activity level of DDC produced by the neuronal progenitor cells is determined by assaying conversion of L-DOPA to dopamine. In some embodiments, the conversion of L-DOPA to dopamine is assayed by adding L-DOPA to the medium after culturing the population of neuronal progenitor cells, and quantifying conversion of the added L-DOPA to dopamine after an incubation period.

In some embodiments, the reference DDC activity level is obtained by obtaining a DDC activity level produced by a population of control cells. In some embodiments, the control cells are pluripotent stem cells, such as embryonic stem cells or induced pluripotent stem cells.

The invention also provides, in some embodiments, a potency assay matrix that includes determining one or more properties such as cell health, optimal maturity/identity, engraftment potential, and dopamine production potential of a population of DANPCs. In some embodiments, the potency assay matrix includes performing the DDC activity assay in combination with one or more of RNAseq-based assays for neuronal cell identity and maturity (e.g., NeuriTest), predicted engraftment of neuronal cells derived from neuronal progenitor cells (e.g., GraftTest), and predicted dopamine production by neuronal cells derived from neuronal progenitor cells after implantation (e.g., DopaTest).

As an example, in some embodiments, the invention provides a potency assay matrix in which a population of neuronal progenitor cells is obtained by culturing pluripotent stem cells under conditions to neurally differentiate the cells to obtain a population of neurally differentiated pluripotent stem cells. The differentiation state of the neurally differentiated pluripotent stem cells is classified by calculating a first similarity score using a test dataset and a first reference dataset, wherein: a) the first reference dataset comprises a representation of gene expression levels for one or more genes that are differentially expressed between cells at a first differentiation state and cells at a differentiation state of a determined dopaminergic neuronal cell; b) the test dataset comprises expression levels for genes that are expressed in one or more test cells comprised in the population of neurally differentiated pluripotent stem cells, wherein the expression levels in the test dataset comprise expression levels for one or more of the genes for which a representation of expression levels are included in the first reference dataset, and c) the first similarity score indicates whether the differentiation state of the test cells is more similar to the first differentiation state or to the differentiation state of a determined dopaminergic neuronal progenitor cell.

In some embodiments, the method further includes classifying the differentiation state of the population of neurally differentiated pluripotent stem cells by calculating a second similarity score using the test dataset and a second reference dataset, wherein: a) the second reference dataset comprises a representation of gene expression levels for one or more genes that are differentially expressed between cells at a differentiation state of a determined dopaminergic neuronal progenitor cell and cells at a third differentiation state; b) the second similarity score indicates whether the differentiation state of the test cells is more similar to the differentiation state of a determined dopaminergic neuronal progenitor cell or to the third differentiation state; and c) classifying the differentiation state of the one or more test cells based on one or both of the first similarity score and the second similarity score.

In some embodiments, the method further includes determining a novelty score which indicates the similarity of cells in the population of population of neurally differentiated pluripotent stem cells to a reference cell population of dopaminergic neuronal progenitor cells.

In some embodiments, the invention provides a potency assay matrix that includes an assay for predicting engraftment capability of the population of neurally differentiated pluripotent stem cells by: a) determining, in a test sample that comprises a population of neuronal progenitor cells, a gene expression level for one or more gene associated with engraftment (G genes), wherein the G genes are selected from the group consisting of AC000120.3, KRT77, TTR, PRR16, MEGF10, PDE3A, GDPD2, CMTM8, APOA1, CMTM7, CDHR3, CORIN, VTN, CPNE8, EFEMP1, CD47, SPARC, JAM2, CDO1, PLXDC2, DYNLL2, ITGA3, RPS6KL1, CHRNB2, SULT4A1, PTPN3, LZTS1, RUNX1T1, TMEM145, EPHA10, CARMIL3, MANEAL, TMEM176B, MPP3, DRAXIN, ADGRB1, KIF26A, CELF5, CNTN2, ASPHD1, SVOP, ANGPT2, SLC22A15, SRRM3, GRIN2D, DACH2, CHST1, GRIN1, LHX5, and NOS2; and b) comparing the gene expression level in the test sample for each G gene of the one or more G genes to a control level for the expression level of the G gene, thereby predicting whether neuronal cells derived from the neuronal progenitor cells are likely to engraft in a brain region of a subject following implantation of the population of neuronal progenitor cells in the brain region.

In some embodiments, the invention provides a potency assay matrix that includes an assay for predicting dopamine production by neuronal cells obtained from a population of neuronal progenitor cells, the method comprising: a) determining, in a test sample that comprises a population of neuronal progenitor cells, a gene expression level for one or more gene associated with dopamine production (D genes), wherein the D genes are selected from the group consisting of CNTNAP5, KLHL1, NHLH2, GREM2, BRINP2, GRIN3A, LRRC4C, IRX3, CPNE4, PTPN3, PMEL, PCDH20, LRRC37A2, TMEM246, B3GALNT1, ZHX1, BCAS4, SLC25A37, GRINA, MID1, FRMD4A, PARP10, WHAMMP2, EYA1, CORO2B, WHAMMP3, B3GALT5, GPR35, ABCD2, ITIH3, AC107464.1, CAMK2N1, CAMK2A, PRPS1, GOLGA6L10, AMOT, SULT1A1, CD83, SPON1, FRMPD3, AC096570.1, TCAF2, GOLGA8M, VWA5B2, CA8, AC017050.1, KRT77, AP000350.6, LINC02751, and ARHGAP5-AS1; and b) comparing the gene expression level in the test sample for each D gene of the one or more D genes to a control level for expression level of the D gene, thereby predicting whether neuronal cells derived from the neuronal progenitor cells are likely to exhibit dopamine production following implantation of the population of neuronal progenitor cells to the subject.

In some embodiments, the potency assay matrix includes performing one or more of the neural identity, engraftment, and dopamine production predictive assays on bulk RNAseq data obtained from a population of neuronally differentiated cells to determine whether the population includes dopaminergic neuronal progenitor cells. If so, the dopaminergic neuronal progenitor cells may then be cryopreserved and, when ready for use, thawed and subjected to the DDC activity assay to determine whether the cells retain sufficient potency for implantation.

In some embodiments, the invention provides methods of preparing a pharmaceutical composition for implantation into a subject in need of treatment for a neurodegenerative disease, wherein the pharmaceutical composition includes a population of neuronal progenitor cells and the method includes: a) culturing a population of neuronal progenitor cells in a suitable medium and b) determining an expression level of L-dihydroxyphenylalanine (DOPA) decarboxylase (DDC) by the population of neuronal progenitor cells wherein an increased expression level of DDC by the population of neuronal progenitor cells compared to a reference DDC expression level indicates that the neuronal progenitor cells are suitable for implantation into the subject. In some embodiments, the neuronal progenitor cells were previously cryopreserved. In some embodiments, the neurodegenerative disease is Parkinson's Disease. In some embodiments, the invention provides methods of treating a neurodegenerative disease in a subject in need thereof by administering a therapeutically effective amount of a pharmaceutical composition prepared by the above method.

In some embodiments, the invention provides methods for obtaining a composition that includes dopaminergic neuronal progenitor cells in which the methods involve a) differentiating a population of pluripotent stem cells under conditions to neurally differentiate the pluripotent stem cells into neuronal progenitor cells, and b) determining an activity level of L-dihydroxyphenylalanine (DOPA) decarboxylase (DDC) in a sample of the neuronal progenitor cells, wherein an increased activity level of DDC in the sample compared to a reference DDC activity level indicates that the neuronal progenitor cells in the sample are dopaminergic neuronal progenitor cells. In some embodiments, the pluripotent stem cells are induced pluripotent stem cells. In some embodiments, the induced pluripotent stem cells are obtained by reprogramming somatic cells obtained from a subject in need of treatment for a neurodegenerative disease such as Parkinson's Disease.

In some embodiments, the invention provides a method of determining potency of a pharmaceutical composition comprising neuronal progenitor cells intended for treating a neurodegenerative disease, in which the methods involve determining an activity level of L-dihydroxyphenylalanine (DOPA) decarboxylase (DDC) in a sample obtained from the pharmaceutical composition, wherein an increased activity level of DDC in the sample compared to a reference DDC activity level is predictive of dopamine production by neuronal cells derived from the neuronal progenitor cells in the pharmaceutical composition. In some embodiments, the pharmaceutical composition includes neuronal progenitor cells that were obtained by differentiating a population of pluripotent stem cells under conditions to neurally differentiate the pluripotent stem cells into neuronal progenitor cells. In some embodiments, the sample is obtained by thawing a previously frozen aliquot of the pharmaceutical composition comprising neuronal progenitor cells. In some embodiments, the sample is obtained by culturing an aliquot of the pharmaceutical composition in a culture medium and the method involves determining an activity level of DDC in the culture medium or in a cell lysate obtained from the cultured aliquot of the pharmaceutical composition. In some embodiments, the expression level of DDC in the sample is determined by assaying conversion of L-DOPA to dopamine. In some embodiments, conversion of L-DOPA to dopamine is assayed by adding L-DOPA to the sample and quantifying conversion of the added L-DOPA to dopamine after an incubation period. In some embodiments, conversion of the added L-DOPA to dopamine is determined by quantitating dopamine in the sample using LD-MS.

### BRIEF DESCRIPTION OF DRAWINGS

FIG. 1 shows the biosynthetic pathway for conversion of tyrosine to dopamine.

FIG. 2 shows the relative gene expression of critical genes at various stages of DANPC maturity. DDC is significantly elevated in drug product DANPCs.

FIG. 3 shows an overview of a bioassay for DDC in which DDC function is quantified by percent conversion of L-DOPA to dopamine.

FIG. 4 shows a test of lot-to-lot variability and assay parameters such as incubation time and L-DOPA concentration.

FIG. 5 shows results of an experiment that characterizes the minimum rest time required to assay DANPCs.

FIG. 6 shows a comparison of DDC activity data from DANPCs and iPSC negative controls that was compiled to characterize the range and sensitivity of the DDC assay.

FIGS. 7A and 7B show results of sensitivity of a test of cell stability by simulating cold temperature storage deviations.

FIG. 8 shows quantification of dopamine released by DANPCs versus iPSCs by normalizing dopamine release to the number of cells in each well.

FIG. 9A shows the calculation of a measure of the maturity and identity of a population of DANPCs using a model that is based on bulk RNAseq data. FIG. 9B shows the calculation of a novelty score which determines whether a putative population of DANPCs actually does contain DANPCs and not cells of another type.

FIG. 10A shows a schematic of an assay for predicting whether neuronal cells derived from a population of neuronal progenitor cells is likely to engraft successfully after implantation. FIG. 10B shows the two models that were explored-Supervised Principal Component Analysis (SPCA) and multiple gene-based estimates. FIG. 10C shows the performance of the top-ranked models.

FIG. 11A shows a schematic of an assay for predicting whether neuronal cells derived from a population of neuronal progenitor cells is likely to produce dopamine after implantation. FIG. 11B shows the two models that were explored-Supervised Principal Component Analysis (SPCA) and multiple gene-based estimates. FIG. 11C shows the performance of the top-ranked models.

### DETAILED DESCRIPTION OF THE INVENTION

The invention provides, in some embodiments, an assay for assessing potency of dopaminergic neuronal progenitor cells (DANPCs). Potency assays are often required for regulatory approval of cell therapy products such as DANPCs. It is often desirable to have a such a potency assay that measures function relative to the proposed mechanism of action of the drug product, particularly when the drug product is in its final form prior to use to treat a subject (e.g., after the drug product has been thawed after cryopreservation). Accordingly, the invention provides, in some embodiments, a potency assay for DANPCs that also provides a measure of cell health.

### Differentiation of Neuronal Progenitor Cells

The invention is useful for determining potency of dopaminergic neuronal progenitor cells (DANPCs) that are, in some embodiments, obtained by differentiating pluripotent stem cells under conditions to neurally differentiate the cells. The pluripotent stem cells are, in some embodiments, embryonic stem cells or induced pluripotent stem cells (IPSCs). In some embodiments, the pluripotent stem cells are autologous to the subject into which the neuronal progenitor cells are to be implanted.

Suitable differentiation methods for obtaining neuronal progenitor cells are known to those of skill in the art. Such methods can involve manipulating gene expression either directly (e.g., by delivery of a genetic payload into a cell) or indirectly (e.g., by using a variety of pharmacological agents to tilt the differentiation pathway towards a neuronal fate. Telias (2023) Neural Regen. Res. 18:1273-1274. The dual-SMAD inhibition protocol is an example of the latter method of neurally differentiating pluripotent stem cells to neuronal progenitor cells (Chambers et al. (2009) Nat. Biotechnol. 27:275-280. This process can involve exposing pluripotent stem cells to (a) an inhibitor of bone morphogenetic protein (BMP) signaling; (b) an inhibitor of TGF-β/activin-Nodal signaling; and (c) at least one activator of Sonic Hedgehog (SHH) signaling. The method can further include exposing the pluripotent stem cells to at least one inhibitor of GSK3β signaling. Methods for neurally differentiating pluripotent stem cells are also described in, for example, US Patent Publication 2023/0059010, entitled METHODS OF DIFFERENTIATING NEURAL CELLS AND RELATED COMPOSITIONS AND METHODS OF USE.

Direct genetic manipulation of stem cells to induce neuronal differentiation can involve introducing a nucleic acid that encodes a neuronal gene, and/or regulatory sequences such as promoters and enhancers. Such methods can involve introducing a nucleic acid construct into the pluripotent stem cells by either (i) plasmid transfection, lipofection, or electroporation, or (ii) use of viral vectors such as adeno-associated virus or lentiviral vectors. Lentiviral-based neuronal differentiation methods are described in, for example, Zhang et al. (2013) Neuron 78:785-798.

In some embodiments, the differentiation conditions are by culture that includes an adherent cell culture, such as using methods as described in, for example, Chambers et al., in which the neuronal progenitor cells are obtained between day 18 and day 24 after initiation of the differentiation process. In some embodiments, the differentiation conditions include suspension cell culture, such as any as described herein and in US Patent Publication 2023/0059010, in which the neuronal progenitor cells are obtained between day 13 and day 20 after initiation of the differentiation process, such as on or about day 16. In some embodiments, the pluripotent stem cells are induced pluripotent stem cells (iPSCs). In some embodiments the media is supplemented with a Rho-associated protein kinase (ROCK) inhibitor on one or more days when cells are passaged.

### Potency Assay Matrix

Cell therapies such as those intended for treating a neurodegenerative disease such as Parkinson's disease often have a complex spectrum of mechanisms. For such therapies, it is desirable to have a potency assay matrix that can quantitatively and accurately characterize the various important functions of the cells after they have matured. The present invention provides a potency assay matrix that includes one or more assays, each of which is directed to determining potency with respect to a different mechanism of action. For example, the potency assay matrix can involve: a) determining potency and cell health of DANPCs using a DDC assay of the invention as described below, b) an assay for determining optimal DANPC identity and maturity, c) an assay for predicting whether neuronal cells derived from the DANPCs will successfully engraft after implantation of the DANPCs into a brain of a subject, and d) an assay for predicting whether neuronal cells derived from the DANPCs will produce dopamine after implantation of the DANPCs into a brain of a subject. In some embodiments, the potency assay matrix will include two or more of these assays; in other embodiments, three or more of the assays are used, and in further embodiments, all four of the assays are used as the potency assay matrix.

### Potency and Cell Health Assays

In some embodiments, the invention provides a potency assay that determines whether a population of DANPCs can produce dopamine, and thus is potent with respect to an important mechanism of action for using the DANPCs to treat a neurodegenerative disease such as Parkinson's disease. The US Food and Drug Administration (FDA), for example, requires that cell therapy products have a potency test, and recommends that the potency test is based on the mechanism of action of the product.

In some embodiments, the invention provides a potency assay that involves determining an amount of L-dihydroxyphenylalanine (DOPA) decarboxylase (DDC) activity produced by a population of neuronal progenitor cells. This assay is directly related to an important mechanism of action that is necessary for successful treatment of a neurodegenerative disease such as Parkinson's disease, and therefore is suitable for meeting regulatory potency requirements such as those set forth by the US FDA and other agencies such as the European Medicines agency and corresponding agencies in other countries.

The assays can involve determining an activity level of DDC produced by a population of DANPCs, wherein an increased activity level of DDC produced by the population of DANPCs compared to a reference DDC activity level indicates that the population has sufficient potency for use to treat a subject in need of treatment of a neurodegenerative disease such as Parkinson's Disease. The provided methods can, some embodiments, be used to aid lot selection of in vitro differentiated neuronal progenitor cells for use as a therapeutic composition and to determine whether a population of DANPCs remains potent after cryopreservation and subsequent thawing.

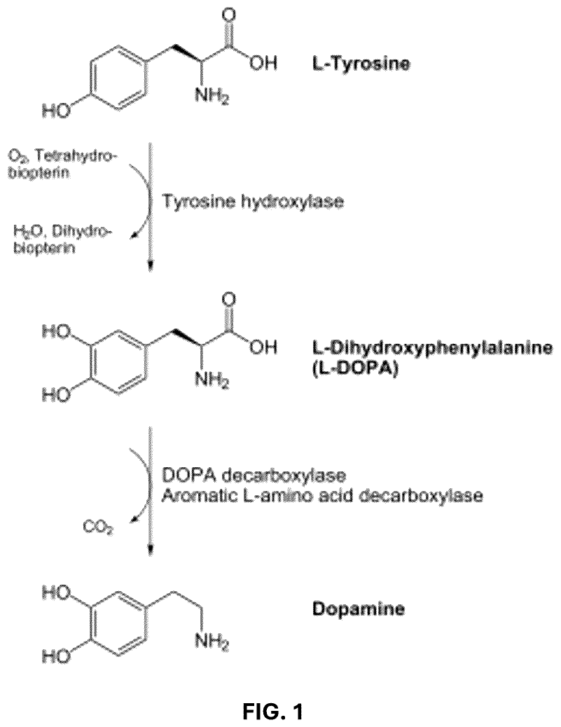

DOPA decarboxylase (DDC), also known as aromatic L-amino acid decarboxylase (AADC), is involved in dopamine biosynthesis. Klein et al. (2019) Cellular and Mol. Neurobiol. 39:31:59. The primary biosynthetic pathway for dopamine is a two-step process in which tyrosine hydroxylase (TH) converts tyrosine to levodopa (L-DOPA) using tetrahydrobiopterin, oxygen, and iron as cofactors (FIG. 1). L-DOPA is then converted to dopamine by DDC, with pyridoxal phosphate as a cofactor. Christenson et al. (1970) Arch. Biochem. Biophys. 141:356-367. A recent report indicates that increased DDC levels in cerebrospinal fluid and plasma is associated with Lewey body disease and Parkinsonian disorders. Pereira et al. (2023) Nature Aging 3:1201-1209. Paradoxically, the present inventors have found that increased DDC activity in neuronal progenitor cells is predictive of increased dopamine production in neuronal cells that are derived from the progenitor cells after implantation.

Several assays for detecting DDC activity are known in the art and are suitable for use in potency assay of the present invention, including high performance liquid chromatography (HPLC) (Hyland and Clayton (1992) Clin. Chem. 38:2405-2410), radiochemical assays (Stevenson et al. (1990) Biochemistry 29:7631-7647), and spectrophotometric assays (Charteris and John (1975) Anal. Biochem. 66:365-371). Another DDC assay that is suitable for use in the present invention uses gold nanoparticles (AuNPs) to detect DDC activity. Park et al. (2013) Analyst 138:3146-3149. This particular assay uses an AuNP aggregation technique. Dopamine can induce a color change of AuNPs from red to blue due to the positively charged amine group of dopamine adsorbing to the negatively charged citrated AuNPs, forming an aggregate of AuNPs. When L-DOPA is used as a substrate for DDC in the presence of AuNPs, however, the AuNP solution is well dispersed and retains its red color due to shielding of the amine group of L-DOPA by its carboxyl group. The color of the AuNP solution depends on the amount of DA, thereby enabling both qualitative and quantitative measurements of DDC activity.

### NeuriTest

Another component of the potency assay matrix provided by the invention is, in some embodiments, is a predictive 2-score model trained on bulk RNAseq data from efficacious samples in animal experiments that is used to determine optimal DANPC identity and maturity. This assay is described in more detail in US Patent Publication No. 2023/0377685, entitled METHODS OF CLASSIFYING THE DIFFERENTIATION STATE OF CELLS AND RELATED COMPOSITIONS OF DIFFERENTIATED CELLS. The first score indicates whether a population of cells has a desired differentiation state, for example, the differentiation state of a determined dopaminergic neuronal progenitor cell. Determining this score involves calculating a first similarity score using a test dataset and a first reference dataset, wherein the first reference dataset comprises a representation of gene expression levels for one or more genes that are differentially expressed between cells at a first differentiation state and cells at the differentiation state of, for example, a determined dopaminergic neuronal progenitor cell. The test dataset includes expression levels for genes that are expressed in one or cells in a population of cells obtained from, for example, neuronal differentiation of pluripotent stem cells. The expression levels in the test dataset include expression levels for one or more of the genes for which a representation of expression levels are included in the first reference dataset, and the first similarity score indicates whether the differentiation state of the test cells is more similar to the first differentiation state or to the differentiation state of a determined dopaminergic neuronal progenitor cell. The method can also involve calculating a second similarity score using the test dataset and a second reference dataset, wherein the second reference dataset comprises a representation of gene expression levels for one or more genes that are differentially expressed between cells at the differentiation state of a determined dopaminergic neuronal progenitor cell and cells at a third differentiation state, the expression levels in the test dataset comprise expression levels for one or more of the genes for which a representation of expression levels are included in the second reference dataset, and the second similarity score indicates whether the differentiation state of the test cells is more similar to the differentiation state of a determined dopaminergic neuronal progenitor cell or to the third differentiation state; and (c) classifying the differentiation state of the one or more test cells based on one or both of the first similarity score and the second similarity score.

The second score in this part of the potency assay matrix is a novelty score, which indicates if gene expression levels of a test cell population resemble those of reference cell population such as, for example, dopaminergic neuronal progenitor cells. For each reference cell population, a weighted correlation of expression levels across the selected genes (those having median CPM greater than 10) to the mean expression levels across reference cell populations was calculated. For correlation calculation, genes were weighted by 1/CV values. Based on these correlation values, a novelty score cutoff was set such that test cell populations with (one minus weighted correlation values) greater than 0.15 would be identified as dissimilar to the reference cell populations, and thus not suitable for use in treating a subject in need of treatment for a neurodegenerative disease such as Parkinson's disease.

### GraftTest

Another component of the potency assay matrix provided by the invention is a method for predicting whether neuronal cells obtained from a population of neuronal progenitor cells is likely to successfully engraft after implantation of the neuronal progenitor cells into the brain of a subject. This assay, which is described in more detail in U.S. patent application Ser. No. 18/946,611, filed Nov. 13, 2024 entitled METHOS OF PREDICTING ENGRAFTMENT CAPABILITY OF DIFFERENTIATED NEURONAL CELLS, involves (a) determining, in a test sample that comprises a population of neuronal progenitor cells, a gene expression level for one or more gene associated with engraftment (G genes), wherein the G genes are selected from the group consisting of AC000120.3, KRT77, TTR, PRR16, MEGF10, PDE3A, GDPD2, CMTM8, APOA1, CMTM7, CDHR3, CORIN, VTN, CPNE8, EFEMP1, CD47, SPARC, JAM2, CDO1, PLXDC2, DYNLL2, ITGA3, RPS6KL1, CHRNB2, SULT4A1, PTPN3, LZTS1, RUNX1T1, TMEM145, EPHA10, CARMIL3, MANEAL, TMEM176B, MPP3, DRAXIN, ADGRB1, KIF26A, CELF5, CNTN2, ASPHD1, SVOP, ANGPT2, SLC22A15, SRRM3, GRIN2D, DACH2, CHST1, GRIN1, LHX5, and NOS2; and (b) comparing the gene expression level in the test sample for each G gene of the one or more G genes to a control level for the expression level of the G gene, thereby predicting whether neuronal cells derived from the neuronal progenitor cells are likely to engraft in a brain region of a subject following implantation of the population of neuronal progenitor cells in the brain region.

### DopaTest

An additional component of the potency assay matrix provided by the invention is a method for predicting whether neuronal cells derived from a population of neuronal progenitor cells will produce dopamine after implantation of the neuronal progenitor cells into a brain of a subject. This assay is described in more detail in U.S. patent application Ser. No. 18/946,698, filed Nov. 13, 2024 entitled METHOS OF PREDICTING DOPAMINE PRODUCTION CAPABILITY OF DIFFERENTIATED NEURONAL CELLS. These methods involve: (a) determining, in a test sample that comprises a population of neuronal progenitor cells, a gene expression level for one or more gene associated with dopamine production (D genes), wherein the D genes are selected from the group consisting of CNTNAP5, KLHL1, NHLH2, GREM2, BRINP2, GRIN3A, LRRC4C, IRX3, CPNE4, PTPN3, PMEL, PCDH20, LRRC37A2, TMEM246, B3GALNT1, ZHX1, BCAS4, SLC25A37, GRINA, MID1, FRMD4A, PARP10, WHAMMP2, EYA1, CORO2B, WHAMMP3, B3GALT5, GPR35, ABCD2, ITIH3, AC107464.1, CAMK2N1, CAMK2A, PRPS1, GOLGA6L10, AMOT, SULT1A1, CD83, SPON1, FRMPD3, AC096570.1, TCAF2, GOLGA8M, VWA5B2, CA8, AC017050.1, KRT77, AP000350.6, LINC02751, and ARHGAP5-AS1; and (b) comparing the gene expression level in the test sample for each D gene of the one or more D genes to a control level for expression level of the D gene, thereby predicting whether neuronal cells derived from the neuronal progenitor cells are likely to exhibit dopamine production following implantation of the population of neuronal progenitor cells to the subject.

The following examples are included for illustrative purposes only and are not intended to limit the scope of the invention.

### EXAMPLES

The following describes specific examples of how one may use the potency assay matrix to determine whether a population of putative dopaminergic neuronal progenitor cells has sufficient potency for use in treating a subject. Examples 2-4 below show that a set of bioinformatics tests performed on bulk RNA sequencing (RNAseq) data of DANPCs can provide an analytical matrix that provides predictive analytics for the optimal maturity/identity, engraftment potential, and dopamine production potential of the DANPCs. After utilizing a large, high-quality training dataset, these bioinformatics tests can reliably predict in vivo maturation, engraftment efficiency and dopamine production of DANPCs post-transplantation. Each of these properties is important for conferring benefit on a patient. It is desirable, however, to also have an assay for potency that, unlike bulk RNAseq analysis, is able to capture the biochemical or physical function of DANPCs post-thaw. Additionally, recent guidance recommends a bioassay of cellular function, necessitating characterization beyond RNA sequencing. Accordingly, we established an enzymatic assay to evaluate DANPC functionality post-thaw.

### Example 1: L-Dihydroxyphenylalanine (DOPA) Decarboxylase (DDC) Assay

This Example describes a conventional enzymatic assay that is useful for determining stability, cell health, and functionality of DANPCs that are intended for use in treating a subject in need of treatment for a neurodegenerative disease such as Parkinson's Disease.

Abbreviations/Definitions

| | |
|---|---|
| DANPC | Dopaminergic Neuron Precursor Cell |
| DP | Drug Product |
| MOA | Mechanism(s) of action |
| DDC | DOPA decarboxylase |
| FDA | Food and Drug Administration |
| RNA | Ribonucleic Acid |
| TH | Tyrosine Hydroxylase |
| L-DOPA | Levodopa |
| RNAseq | RNA sequencing |
| LAT1 (SLC7A5) | L-type Amino Acid Transporter 1 |
| 4F2hc (SLC3A2 or CD98) | 4F2 cell-surface antigen heavy chain |
| iPSC | Induced Pluripotent Stem Cell |
| VMAT2 | Vesicular Monoamine Transporter 2 |
| FITC | Fluorescein isothiocyanate |
| LC-MS | Liquid chromatography-mass spectrometry |
| ULOQ | Upper limit of quantification |
| LOD | Limit of Detection |
| v/v | Volume/volume percentage |
| ROCK Inhibitor | Rho Kinase Inhibitor |
| HBSS | Hanks' Balanced Salt Solution |

### Methodology

Neuronal progenitor cells were obtained by first converting dermal fibroblasts obtained from multiple donors into iPSCs. The resulting iPSCs were then subjected to neuronal differentiation to obtain neuronal progenitor cells. At Day 20 after initiation of the differentiation process, the cells were cryopreserved.

The neuronal progenitor cells were thawed, resuspended in complete Maturation Media with ROCK Inhibitor, and plated in Laminin, Fibronectin and Poly-L-Ornithine coated 24 well plates at 1.9×10⁶ cells per well. To allow the cells to recover and adhere to the plate, the cells were rested at 37° C. and 5% CO₂ for 1-4 days and fed complete Maturation Media daily prior to assay. Control iPSCs were EDTA passaged or thawed into Laminin 511e8 (recombinant Laminin E8 fragment) coated plates with mTeSR1 media and fed daily until approximately 80% confluent.

On the day of the assay, cell culture media was removed and wells were washed with 500 L HBSS. After removing the HBSS wash, 250 μL HBSS containing 0-5 μM of L-DOPA was added to each well. After 1-4 hours at 37° C. and 5% CO₂, 50 μL of incubation media was collected and added to 50 μL stabilization buffer (50 mM L-Ascorbic Acid in 25% methanol/H20, 1:1 v/v). After collecting supernatant samples in triplicate, cells were washed with HBSS and collected for cell lysates by scraping in 0.5 mL HBSS and transferring to a tube. The wells were washed again with another 0.5 mL HBSS and combined, after which samples were centrifuged, supernatant removed, and pellets snap frozen. Samples were then subjected to LC-MS/MS analysis of dopamine and L-DOPA content.

### Results and Calculations

### Identification of DDC as a Candidate for Bioassay

Functional assessment for the purpose of potency should be assayed on the drug product itself. This requires identifying a mechanism of action-specific relevant function that DANPCs can perform immediately after thawing. Assay of tyrosine hydroxylase (TH)+ neurons and dopamine release requires that DANPCs be matured for extended periods of time, so these are not suitable for potency assessment of the actual drug product (e.g., DANPCs that had been cryopreserved and thawed).

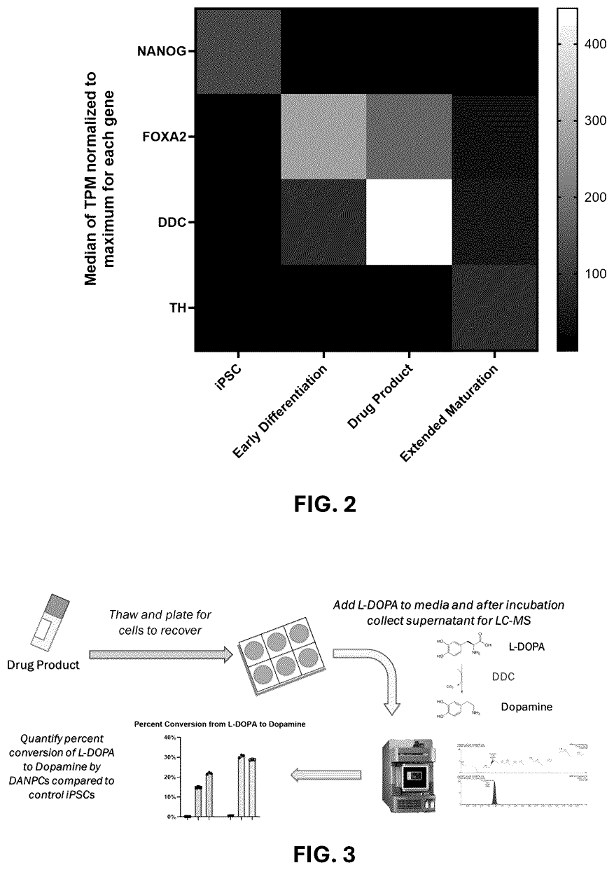

By analyzing relative gene expression of critical genes at various stages of DANPC maturity, we found that DDC expression is significantly elevated in DANPCs as compared to iPSCs at Day 20 (FIG. 2). DDC converts L-Dihydroxyphenylalanine (L-DOPA) into dopamine (FIG. 1), which is a critical step in the function of dopamine producing neurons. The theoretical mechanism of a DDC activity assay is that L-DOPA added to cell culture media is transported into the cytosol via L-type amino acid transporter 1 (LAT1 or SLC7A5). Cytosolic L-DOPA is converted to dopamine via DDC. Cytosolic dopamine is collected into vesicles via vesicular monoamine transporter 2 (VMAT2). Vesicles release the dopamine into the cell culture media, which is then collected and stored in stability buffer until analysis.

### DDC Assay

FIG. 3 shows an overview of the DDC bioassay component of the potency assay matrix. In this embodiment, DDC function is quantified by percent conversion of L-DOPA to dopamine. DANPCs were thawed and rested to recover and adhere to the plate. On the day of the assay, cell culture media was removed and cells were washed. Buffer containing L-DOPA was added to each well and after a short incubation, incubation media was collected and added stabilization buffer. Samples were analyzed by LC-MS/MS for dopamine quantification.

To test lot to lot variability and assay parameters such as incubation time and L-DOPA concentration, DANPCs from 4 donors were assayed for short or long incubations with varying concentrations of L-DOPA. Results, shown in FIG. 5, demonstrate that supernatant media from all lots of DANPCs contained significant levels of DDC activity, while iPSC control supernatant media did not contain DDC activity.

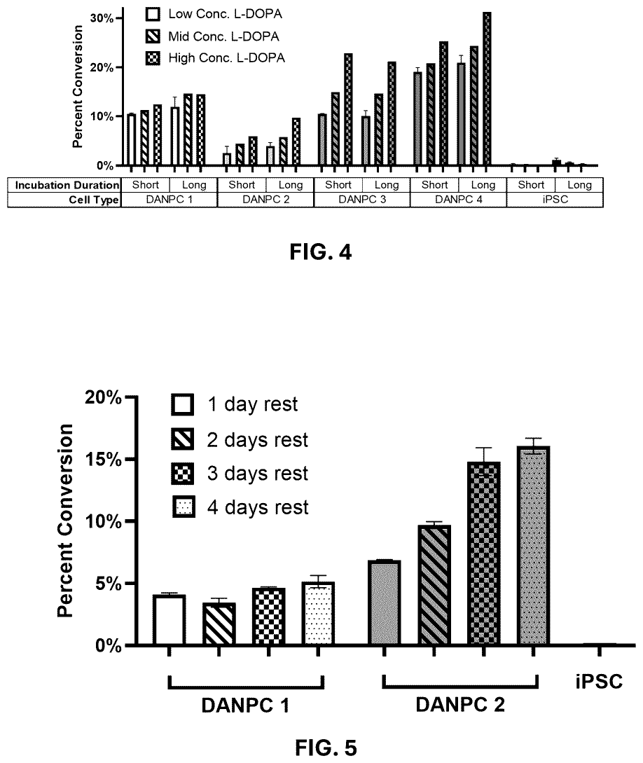

We then characterized the minimum rest time required to assay DANPCs by measuring DDC activity after 1-4 days of rest post-thaw. As shown in FIG. 4, DDC activity was measurable after a single day of rest.

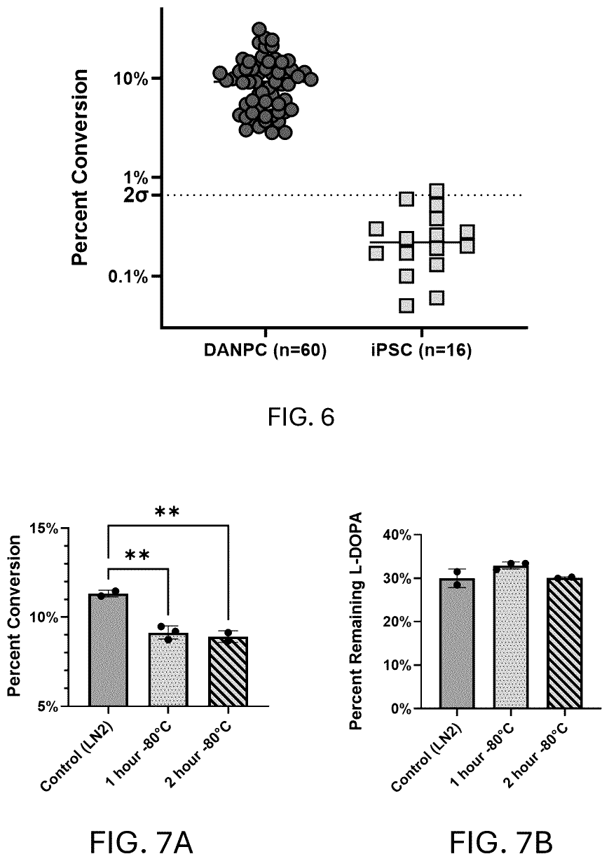

To characterize the range and sensitivity of the assay, we analyzed DDC activity data from DANPCs and iPSC negative controls (FIG. 6) and Table 1 below.

TABLE 1

| | DANPC | iPSC |
|---|---|---|
| Number of values | 60 | 16 |
| Minimum | 2.82% | 0.05% |
| Maximum | 31.3% | 0.73% |
| Mean | 10.13% | 0.27% |
| Std. Deviation | 6.21% | 0.19% |
| Std. Error of Mean | 0.80% | 0.049% |

To determine the sensitivity of the DDC assay to cold storage temperature conditions, we moved DANPCs from LN2 to −80 C storage for 1 or 2 hours. DANPCs that underwent temperature deviations had significantly reduced percent conversion of L-DOPA (FIG. 7A). All conditions had similar remaining percentages of L-DOPA, indicating that the difference in percent conversion was a result of DDC activity and not L-DOPA uptake (FIG. 7B).

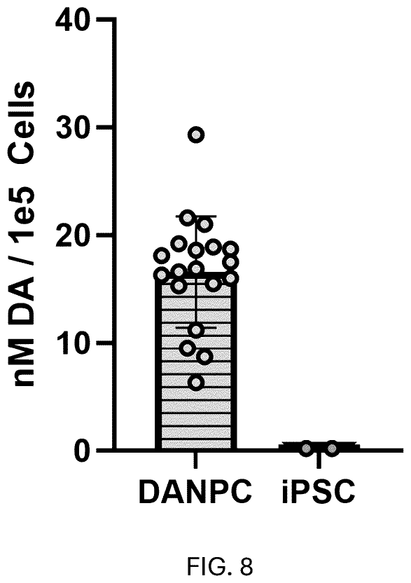

Normalizing release of dopamine to the number of cells in each well is another method to quantify DDC activity instead of calculating percent conversion. Following collection of supernatant after incubation with L-DOPA, cells were collected from the wells assayed and viable cells counted. The concentration of dopamine released by DANPCs was normalized to nM of dopamine per 10⁵ cells and compared to iPSC controls, demonstrating greater release of dopamine by DANPCs (mean 8.48 nM DA/1e5 cells, Std. Error of Mean 0.46 nM DA/1e5 cells, n=43) compared to iPSCs (mean 0.21 nM DA/1e5 cells, Std. Error of Mean 0.02 nM DA/1e5 cells, n=6) (FIG. 8).

In conclusion, this example demonstrates that DDC activity of freshly thawed DANPCs can be assayed as a traditional enzymatic test of functional activity. DANPCs only require an overnight rest prior to assaying, release dopamine at a significant level above iPSC negative controls, and DDC activity is a stability sensitive measurement.

### Example 2: NeuriTest

NeuriTest is a predictive 2-score model trained on bulk RNAseq data from efficacious samples in animal experiments and to target optimal DANPC identity and maturity. NeuriTest is described in, for example, US Patent Publication No. 2023/0377685, entitled METHODS OF CLASSIFYING THE DIFFERENTIATION STATE OF CELLS AND RELATED COMPOSITIONS OF DIFFERENTIATED CELLS.

A sample of each DANPC lot was used for RNA extraction, cDNA synthesis, amplification, bulk RNA sequencing using 150 bp PE on an Illumina platform.

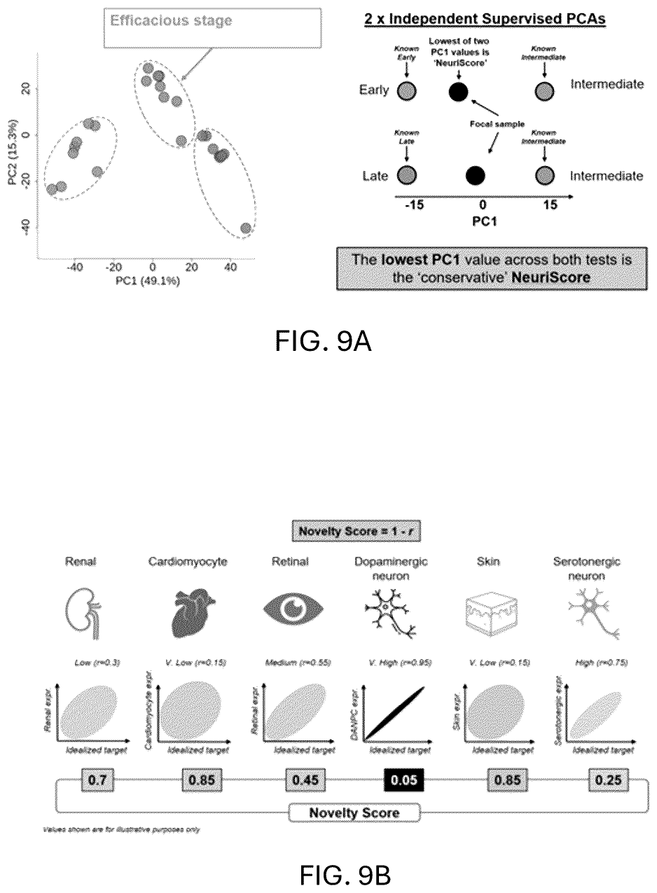

The first score is a measure of the appropriate maturity of a DANPC sample (NeuriScore) (FIG. 9A), in which we identified genes with differential expression between DANPCs of different maturity. The lowest PC1 value, indicating the greatest similarity to either the “Early” or “Late” state, across both tests is used as the NeuriScore, ensuring the most conservative result is reported. The second score, shown in FIG. 9B, is the global, transcriptome wide (~11,500 genes) similarity of the sample to an idealized DANPC sample (Novelty Score). This score indicates whether the cells in the population are actually DANPCs and not cells of a different type (e.g., cardiomyocytes, etc.). To illustrate the Novelty Score, non-DANPC example cell identities are also shown.

### Example 3: GraftTest

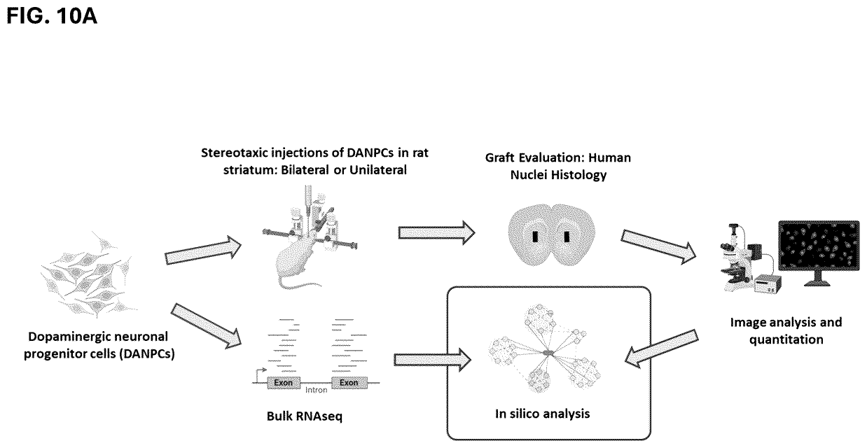

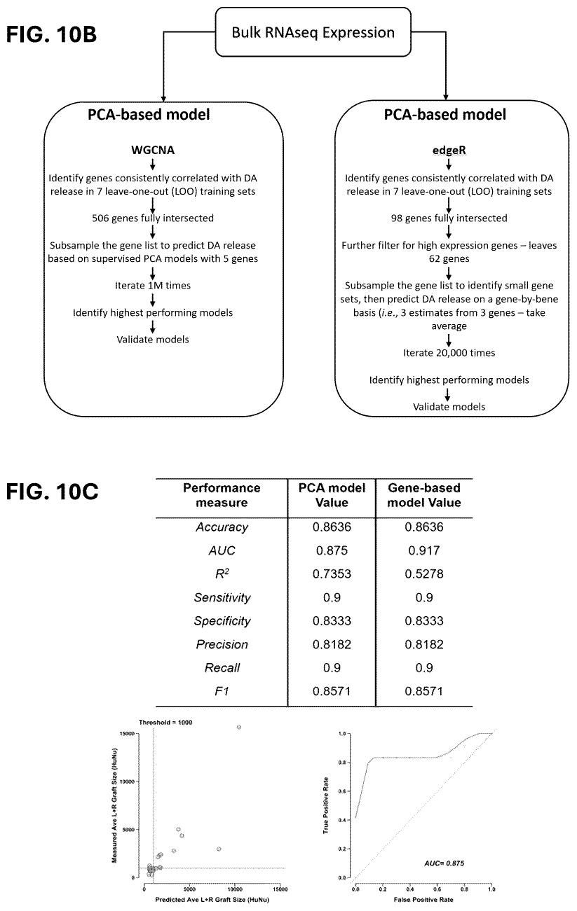

GraftTest is a model that uses bulk RNAseq data and was trained on in vivo data to predict engraftment after neuronal progenitor cells are implanted into a subject. To generate a predictive model of engraftment, DANPCs were transplanted into the striatum of RNU rats, and following engraftment brains were collected and sectioned for histological analysis of human nuclei content (FIG. 10A). A cell suspension of 450k DANPCs was transplanted unilaterally or bilaterally into naïve or 6-OHDA-lesioned striatum of RNU rats. Brain tissues were collected three weeks or months later and stored until processing. Brain samples were sectioned at 40 μm and 1 in 6 sections were used for each IHC procedure. Tissue sections were mounted on positive charged glass slides, dehydrated and cover-slipped before imaging with an Olympus VS200 slide scanner. 20× images were screened for high quality resolution and were imported into Visiopharm software, an AI-based image analysis tool. The number of surviving cells were evaluated by the total count of human nuclei in each graft using an APP developed to perform nuclear segmentation and automatic counts of nuclei for each striatal hemisphere. Graft quantifications were paired with bulk DANPC RNAseq data for in silico analysis and model building.

Two main model types were explored, including Supervised Principal Components Analysis (SPCA) and multiple gene-based estimates (FIG. 10B). In both approaches, genes with inherent correlations with graft size were the features of interest, and the outcome variables were human nuclei counts provided by image analysis The top ranked models (by Accuracy, then AUC) showed similar performance with a small trade-off between the R2 and AUC measures based on validation on in-house data (FIG. 10C).

### Example 4: DopaTest

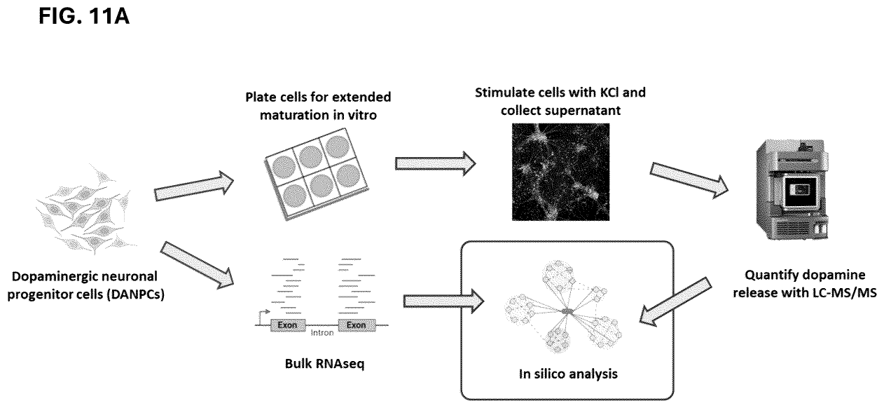

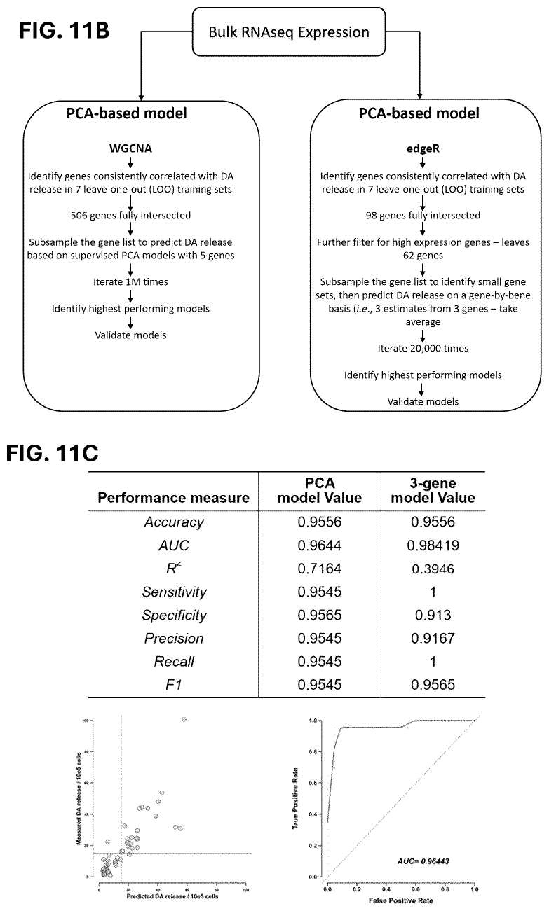

DopaTest is a model that uses bulk RNAseq data and was trained on in vivo data to predict dopamine production after neuronal progenitor cells are implanted into a subject. To generate predictive model of dopamine release, DANPCs were thawed and plated for extended in vitro maturation. After approximately 60 days, cell culture media was removed and buffer containing KCl or a buffer alone was applied to the cells. Supernatant was removed, placed in stability buffer, and frozen prior to analysis. Supernatant was assayed by LC-MS/MS for dopamine and serotonin released in response to KCL stimulation. Dopamine measurements were paired with bulk DANPC RNAseq for in silico analysis and model building (FIG. 11A). Two main model types were explored, including SPCA and multiple gene-based estimates (FIG. 11B). In both approaches, genes with inherent correlations with dopamine production were the features of interest, and the outcome variables were in vitro dopamine release. The top ranked models (by Accuracy, then AUC) showed similar performance with a small trade-off between the R2 and AUC measures based on validation on in-house data (FIG. 11C).

### Example 5: Potency Assay Matrix

Examples 2-4 show that a set of bioinformatics tests performed on bulk RNA sequencing (RNAseq) data of DANPCs can provide an analytical matrix that provides predictive analytics for the optimal maturity/identity, engraftment potential, and dopamine production potential of the DANPCs. After utilizing a large, high-quality training dataset, these bioinformatics tests can reliably predict in vivo maturation, engraftment efficiency and dopamine production of DANPCs post-transplantation. Each of these properties is important for conferring benefit on a patient. It is desirable, however, to also have an assay for potency that, unlike bulk RNAseq analysis, is able to capture the biochemical or physical function of DANPCs post-thaw. Additionally, recent guidance recommends a bioassay of cellular function, necessitating characterization beyond RNA sequencing.

The stability-indicating, conventional, enzymatic DDC bioassay of the functionality of DANPCs as described in Example 1, in combination with the predictive bulk RNAseq-based bioinformatics tests of Examples 2-4, provides a robust analytical development strategy for Parkinson's Disease cell replacement therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

1. A method of assessing potency of a population of neuronal progenitor cells intended for implantation into a subject in need of treatment for a neurodegenerative disease, the method comprising:
assessing potency of the population of neuronal progenitor cells by determining an L-dihydroxyphenylalanine (DOPA) decarboxylase (DDC) activity level produced by the neuronal progenitor cells;
wherein a DDC activity level that is elevated relative to that produced by a population of reference cells is indicative of sufficient potency for implantation.

2. The method of claim 1, wherein the neuronal progenitor cells are cultured in a suitable medium before determining the DDC activity level.

3. The method of claim 1, further comprising releasing the population of neuronal progenitor cells for therapeutic use if the assessed potency meets a regulatory potency requirement for release.

4. The method of claim 3, wherein the regulatory potency requirement(s) for release are as determined by the US Food and Drug Administration, the European Medicines Agency, or the Japanese Pharmaceuticals and Medical Devices Agency.

5. The method of claim 2, wherein the method comprises determining the activity level of DDC in the medium after culturing the population of neuronal progenitor cells.

6. The method of claim 1, wherein the method comprises determining the activity level of DDC in a cell lysate obtained from the population of neuronal progenitor cells.

7. The method of claim 1, wherein the DDC activity level of the population of neuronal progenitor cells is determined by assaying conversion of L-DOPA to dopamine.

8. The method of claim 7, wherein conversion of L-DOPA to dopamine is assayed by adding L-DOPA to the medium after culturing the population of neuronal progenitor cells, and quantifying conversion of the added L-DOPA to dopamine after an incubation period.

9. The method of claim 1, wherein the reference DDC activity level is obtained by determining a DDC activity level produced by a population of control cells.

10. The method of claim 9, wherein the control cells comprise pluripotent stem cells.

11. The method of claim 1, wherein the population of neuronal progenitor cells is obtained by culturing pluripotent stem cells under conditions to neurally differentiate the cells to obtain a population of neurally differentiated pluripotent stem cells.

12. The method of claim 11, wherein the method further comprises classifying the differentiation state of the neurally differentiated pluripotent stem cells by calculating a first similarity score using a test dataset and a first reference dataset, wherein:
a) the first reference dataset comprises a representation of gene expression levels for one or more genes that are differentially expressed between cells at a first differentiation state and cells at a differentiation state of a determined dopaminergic neuronal cell;
b) the test dataset comprises expression levels for genes that are expressed in one or more test cells comprised in the population of neurally differentiated pluripotent stem cells, wherein the expression levels in the test dataset comprise expression levels for one or more of the genes for which a representation of expression levels are included in the first reference dataset, and
c) the first similarity score indicates whether the differentiation state of the test cells is more similar to the first differentiation state or to the differentiation state of a determined dopaminergic neuronal progenitor cell.

13. The method of claim 12, wherein the method further comprises classifying the differentiation state of the population of neurally differentiated pluripotent stem cells by calculating a second similarity score using the test dataset and a second reference dataset, wherein:
a) the second reference dataset comprises a representation of gene expression levels for one or more genes that are differentially expressed between cells at a differentiation state of a determined dopaminergic neuronal progenitor cell and cells at a third differentiation state;
b) the second similarity score indicates whether the differentiation state of the test cells is more similar to the differentiation state of a determined dopaminergic neuronal progenitor cell or to the third differentiation state; and
c) classifying the differentiation state of the one or more test cells based on one or both of the first similarity score and the second similarity score.

14. The method of claim 12, wherein the method further comprises determining a novelty score which indicates the similarity of cells in the population of population of neurally differentiated pluripotent stem cells to a reference cell population of dopaminergic neuronal progenitor cells.

15. The method of claim 11, wherein the method further comprises predicting engraftment capability of the population of neurally differentiated pluripotent stem cells by:
a) determining, in a test sample that comprises a population of neuronal progenitor cells, a gene expression level for one or more gene associated with engraftment (G genes), wherein the G genes are selected from the group consisting of AC000120.3, KRT77, TTR, PRR16, MEGF10, PDE3A, GDPD2, CMTM8, APOA1, CMTM7, CDHR3, CORIN, VTN, CPNE8, EFEMP1, CD47, SPARC, JAM2, CDO1, PLXDC2, DYNLL2, ITGA3, RPS6KL1, CHRNB2, SULT4A1, PTPN3, LZTS1, RUNX1T1, TMEM145, EPHA10, CARMIL3, MANEAL, TMEM176B, MPP3, DRAXIN, ADGRB1, KIF26A, CELF5, CNTN2, ASPHD1, SVOP, ANGPT2, SLC22A15, SRRM3, GRIN2D, DACH2, CHST1, GRIN1, LHX5, and NOS2; and
b) comparing the gene expression level in the test sample for each G gene of the one or more G genes to a control level for the expression level of the G gene, thereby predicting whether neuronal cells derived from the neuronal progenitor cells are likely to engraft in a brain region of a subject following implantation of the population of neuronal progenitor cells in the brain region.

16. The method of claim 11, wherein the method further comprises predicting dopamine production by neuronal cells obtained from a population of neuronal progenitor cells, the method comprising:
a) determining, in a test sample that comprises a population of neuronal progenitor cells, a gene expression level for one or more gene associated with dopamine production (D genes), wherein the D genes are selected from the group consisting of CNTNAP5, KLHL1, NHLH2, GREM2, BRINP2, GRIN3A, LRRC4C, IRX3, CPNE4, PTPN3, PMEL, PCDH20, LRRC37A2, TMEM246, B3GALNT1, ZHX1, BCAS4, SLC25A37, GRINA, MID1, FRMD4A, PARP10, WHAMMP2, EYA1, CORO2B, WHAMMP3, B3GALT5, GPR35, ABCD2, ITIH3, AC107464.1, CAMK2N1, CAMK2A, PRPS1, GOLGA6L10, AMOT, SULT1A1, CD83, SPON1, FRMPD3, AC096570.1, TCAF2, GOLGA8M, VWA5B2, CA8, AC017050.1, KRT77, AP000350.6, LINC02751, and ARHGAP5-AS1; and
b) comparing the gene expression level in the test sample for each D gene of the one or more D genes to a control level for expression level of the D gene, thereby predicting whether neuronal cells derived from the neuronal progenitor cells are likely to exhibit dopamine production following implantation of the population of neuronal progenitor cells to the subject.

17. The method of claim 11, wherein the population of neuronal progenitor cells was cryopreserved after neural differentiation and thawed prior to culturing the neuronal progenitor cells in the medium.

18. The method of claim 11, wherein the pluripotent stem cells are induced pluripotent stem cells (iPSCs) or embryonic stem cells.

19. The method of claim 11, wherein the differentiation conditions comprise adherent cell culture and the composition of neuronal progenitor cells is obtained between day 18 and day 24 after initiation of the differentiation process.

20. The method of claim 11, wherein the differentiation conditions comprise suspension cell culture and the composition of neuronal progenitor cells is obtained between day 13 and day 20 after initiation of the differentiation process.

21. The method of claim 11, wherein the pluripotent stem cells are iPSCs that are obtained by reprogramming somatic cells obtained from a subject in need of treatment for a neurodegenerative disease.

22. A method of preparing a pharmaceutical composition for implantation into a subject in need of treatment for a neurodegenerative disease, the method comprising:
a) culturing a population of neuronal progenitor cells in a suitable medium; and
b) assessing potency of the population of neuronal progenitor cells by determining an L-dihydroxyphenylalanine (DOPA) decarboxylase (DDC) activity level produced by the neuronal progenitor cells;
wherein an elevated DDC activity level relative to a population of reference cells is indicative of sufficient potency use in the pharmaceutical composition.

23. The method of claim 22, wherein the method further comprises cryopreserving the neuronal progenitor cells prior to step (a).

24. The method of claim 22, wherein the neurodegenerative disease is Parkinson's Disease.

25. A method of treating a neurodegenerative disease in a subject in need thereof by administering a therapeutically effective amount of a pharmaceutical composition prepared by the method of claim 22.

26. The method of claim 25, wherein the neurodegenerative disease is Parkinson's Disease.

27. A method of obtaining a composition that comprises dopaminergic neuronal progenitor cells, the method comprising:
a) differentiating a population of pluripotent stem cells under conditions to neurally differentiate the pluripotent stem cells into neuronal progenitor cells; and
b) determining an L-dihydroxyphenylalanine (DOPA) decarboxylase (DDC) activity level produced by the neuronal progenitor cells;
wherein a DDC activity level in the sample is elevated compared to a reference DDC activity level indicates that the neuronal progenitor cells in the sample are dopaminergic neuronal progenitor cells.

28. The method of claim 27, wherein the pluripotent stem cells are induced pluripotent stem cells (iPSCs).

29. The method of claim 28, wherein the induced pluripotent stem cells are obtained by reprogramming somatic cells obtained from a subject in need of treatment for a neurodegenerative disease.

30. The method of claim 29, wherein the neurodegenerative disease is Parkinson's Disease.

31. A method of determining potency of a pharmaceutical composition comprising neuronal progenitor cells intended for treating a neurodegenerative disease, the method comprising determining an activity level of L-dihydroxyphenylalanine (DOPA) decarboxylase (DDC) in a sample obtained from the pharmaceutical composition, wherein an increased activity level of DDC in the sample compared to a reference DDC activity level is predictive of dopamine production by neuronal cells derived from the neuronal progenitor cells in the pharmaceutical composition.

32. The method of claim 31, wherein the pharmaceutical composition comprises neuronal progenitor cells that were obtained by differentiating a population of pluripotent stem cells under conditions to neurally differentiate the pluripotent stem cells into neuronal progenitor cells.

33. The method of claim 31, wherein the sample is obtained by thawing a previously frozen aliquot of the pharmaceutical composition comprising neuronal progenitor cells.

34. The method of claim 31, wherein the sample is obtained by culturing an aliquot of the pharmaceutical composition in a culture medium and the method comprises determining an activity level of DDC in the culture medium or in a cell lysate obtained from the cultured aliquot of the pharmaceutical composition.

35. The method of claim 31, wherein the expression level of DDC in the sample is determined by assaying conversion of L-DOPA to dopamine.

36. The method of claim 35, wherein conversion of L-DOPA to dopamine is assayed by adding L-DOPA to the sample and quantifying conversion of the added L-DOPA to dopamine after an incubation period.

37. The method of claim 36, wherein conversion of the added L-DOPA to dopamine is determined by quantitating dopamine in the sample using LD-MS.
